(19) United States
(12) Patent Application Publication
(10) Pub. No.: US 2026/0130927 A1
(43) Pub. Date: May 14, 2026

(54) PERMEABILITY ENHANCED ORAL THERAPY DELIVERY SYSTEM, AND METHOD OF MANUFACTURE

(71) Applicant: Scott Alan Stelting, Clearwater, FL (US)

(72) Inventor: Scott Alan Stelting, Clearwater, FL (US)

(21) Appl. No.: 18/948,267

(22) Filed: Nov. 14, 2024

(51) Int. Cl. A61K 31/00; A61K 47/02; A61K 47/12; A61K 47/18; A61K 47/44

(52) U.S. Cl. CPC A61K 31/658; A61K 47/02; A61K 47/12; A61K 47/183; A61K 47/44

(57) ABSTRACT

An improved oral therapy delivery system with enhanced permeability, and an innovative method of manufacture. The therapy delivery system is comprised of a ternary, oil in water emulsion containing three (3) phases; a first aqueous phase that is hydrophilic, a second oil phase that is lipophilic, and a third activation or permeability enhancement phase. This three-phase construction allows delivery of both water soluble and oil soluble compounds, with increased activation of either or both. The third activation phase, in some embodiments, is comprised of 4 cellularly vital compounds capable of triggering active transport of material adjacent to cellular surfaces. These 4 compounds are vital for cellular functioning as incentives to trigger active transport of medicinal material.

### TECHNICAL FIELD OF THE INVENTION

The present invention relates to improved delivery systems for medications and more specifically for an oral delivery system for therapies, in an emulsion format.

### BACKGROUND OF THE INVENTION

Delivery of therapies, typically for treatments of a variety of medical problems and ailments, or to help a person achieve any number of improvements of condition, affects the quality of life of millions, perhaps hundreds of millions, of people. A therapy is often a medical treatment for an impairment, disease, disorder, or injury.

A majority of therapies that are delivered to a client or customer are typically delivered in an oral form, including by solid, semi-solid, or liquid form. Examples can include by tablet, capsule, and pill, powder form, or emulsion. Herein, the term “pill” should be understood to mean pills, tablets, capsules, or similar oral delivery system in the art. These types of delivery systems entail a number of problems that limit effectiveness. For one, taking a handful of pills, tablets, or capsules every day, day after day after day, on perhaps multiple occasions per day, can cause “pill fatigue” or “high pill burden.” These are conditions that can occur when a person becomes overwhelmed by the number of medications they have been prescribed and/or the frequency with which they are expected to take them. The act of swallowing a handful of pills is rather unnatural and undesirable, and can be an uncomfortable challenge for some individuals. Taking pills can range from annoying to severely uncomfortable for patients. All this can result in patient reluctance to continue therapies at a high frequency, leading to patient non-compliance. In other words, because of things like pill fatigue or high pill burden, patients may simply stop taking therapies. This can clearly result in the problematic re-emergence of conditions these therapies were supposed to be taken for. Clearly, if a person no longer takes medication for specific health issues, these issues are clearly likely to begin getting worse.

Therapies such as pills, tablets, capsules, or the like can also both have a severe time delay between taking them and effect, and be inefficient with severely limited overall absorption of the therapy.

These therapies such as pills must pass through the gastric tract, breaking down and emptying as they travel through the small and/or large intestine to their eventual delivery destination site for absorption. This contributes to making their reaction significantly time delayed. The duration between taking these powder and pill therapies and the active compounds being absorbed can be 45 minutes up to perhaps hours. This length of time can depend on a variety of factors. These gastric limitations are one of the factors that help make desired therapeutic effect or onset both slow and inefficient.

The desired therapeutic effect is additionally inefficient due to the bioavailability (i.e., absorbability by the body) of the individual therapeutic or medication, and the biological conditions of the individual consumer. The absorption of these therapies, such as pills, and powders through the cells and mucosa (i.e., soft tissue that lines the body's canals and organs in, among others, the respiratory and digestive system) of the consumer, and the resultant pharmacokinetics, is passive. These pills and powders do not activate the cells and mucosa. The passive interaction between the therapies and cells and mucosa tends to result in an inefficient and delayed process.

A rule of thumb for absorption and bioavailability for active ingredients of a therapy delivered as a solid dosage, such as a pill, is between near zero bioavailability to perhaps 20 percent bioavailability for therapies like tablets, capsules, pill, and powders. Following a Pareto distribution, 80 percent or more of the active ingredients are not utilized and pass through the system. Another way to put this is that to have a single dose absorbed and functioning within a person, that person typically has to take five times the dose, with four of the five doses expected to simply not be absorbed, pass through, and be wasted. This low absorption of active ingredients typically results in low efficacy of therapeutics, a slow onset of therapeutic effects, and can be a factor in pill fatigue and patient non-compliance.

To improve this situation, to increase the availability, absorption, and speed of onset, a drinkable delivery system can be used. Currently, there are drinkable (i.e., oral) emulsion delivery systems that also rely on passive delivery. An emulsion is a mixture of liquids that do not dissolve in each other and have droplets of one liquid scattered throughout the other. A well-known example of an emulsion is oil and water. Therapies tend to be hydrophilic (i.e., aqueous) or lipophilic (i.e., oil-based, aka, “lipid-loving”).

Turning to FIG. 1, a typical two-phase nano-emulsion delivery system is shown in use. A therapy cannot normally be delivered into the cells because their forming particles are too large and have no other way to penetrate the semi-permeable cell walls and into the cells, where they can work.

Currently, this is usually addressed by breaking drinkable therapy emulsion particles down into micro-particles or nanoparticles 50. In this representation, nanoparticles are shown. As shown in the figure, when the nanoparticles are small enough, they can passively move through the semi-permeable walls 46 of the cell 44. The very small, nano-emulsion or micro-emulsion particles of these emulsions travel through the body, Including to un-activated cells and mucosa. In these delivery systems, the size of the emulsion particle influences bioavailability. The smaller the particle, the more likely it will be absorbed through the cells or mucosa.

As no work is done or system employed, this movement into the cells is passive, passively absorbed by the cell 44, and represented by arrow set A. Herein the cell and walls are indicated by a representative cell and walls 44, 46, and nanoparticles are indicated by representative nanoparticles 50.

To break emulsion particles down into absorbable nanoparticles, surfactants are typically used. Surfactants both break any oil particles into smaller pieces and lower the surface tension of water. To use an analogy, using a surfactant is like taking a chainsaw to the oil and water components. However, surfactants are basically soap components. When within an orally delivered substance, surfactants, similar to soap in general, taste terrible. Accordingly, simply placing a surfactant within an oral therapy would make a user much less likely to take it, partly defeating the purpose.

Often, this problem is bypassed by putting the emulsion and surfactant into gel caps. However, this bypasses any absorption around the oral cavity, tongue, stomach, anywhere until the gel cap reaches the intestines. This makes the therapy much less effective, also partly defeating the purpose of an oral therapy.

These current emulsion systems, as with other delivery systems, that typically rely on passive absorption of the active ingredients based on particle size, have similar drawbacks to other passive therapies herein, such as limited absorption and time delays in absorption.

Further, current emulsion systems, including those with cannabinoid-based emulsion components, tend to be somewhat sensitive to work with or handle. Because the oil and water components are under constant internal pressures to separate, they tend to have weaknesses of instability, a tendency to separate, inability to be pasteurized, inability to endure freeze-thaw cycles without separating or coming apart, and a need for significant reformulation work for different active ingredients. These are issues whether the emulsion is water-or oil-soluble, or both.

Another issue with therapies, particularly emulsions, is that of contamination. With numerous microorganisms and microbes, such as bacteria and fungi in the air, contamination of a therapy is fairly likely. To prevent this problem, chemical preservatives are currently used. However, these preservatives tend to be unhealthy, have associated costs, and are of limited effectiveness. They are, accordingly, clearly far less than ideal. A more natural method of preservation of such emulsions is much needed.

An important concept to the invention herein for preservation is water activity. Water activity (i.e., a_{w}) is a term of physics used in food science. The water activity (a_{w}) of a food is defined as the ratio between the vapor pressure of the food itself, when in an undisturbed balance with the surrounding air, and the vapor pressure of distilled water under identical conditions. A water activity of 0.80, for example, means the vapor pressure of the food is 80 percent of that of pure water. The water activity increases with temperature (https://www.fda.gov).

Water activity as a term in physics exists on a logarithmic scale of 0.00-1.00. In other terms, water activity is the potential or ability of the water within a substance to be available to interact with other substances. At a water activity of zero, the water is physically tied up and none of it is available for work. At a water activity of 1.0, all the water is available for work or interaction (i.e., 100 percent).

Most foods tend to have a water activity (a_{w}) of 0.95 or higher. This means most associated water is available for interaction and will provide sufficient moisture to support the growth of microbes and microorganisms like bacteria and fungi, such as mold, mildew, and yeasts.

Conversely, the amount of available moisture to interact with can be reduced to a point which will inhibit the growth of the organisms. Lower water activity substances tend to support fewer microbes and microorganisms since these get desiccated by lack of a needed aqueous environment. Microbes and microorganisms typically can't grow at a water activity below 0.75; at 0.70 or less there appears no water available to interact or do work.

As an example, if there is a solution of water with a high amount of salt, the activity is very low, possibly near 0.00, because the water is already interactive with the salt. Accordingly, fresh water tends to have more water activity than salt water. At the ocean, a saltwater breeze lowers water activity relatively. This is why a saltwater breeze, and areas near the ocean, often feels relatively cooler than it really is. Lower water activity feels cooler than higher water activity.

If water activity of a therapy delivery system can be reduced below 0.70, then preservatives would not be needed. However, a way to successfully implement this into a therapy delivery system does not appear to currently exist.

What is needed is a therapy delivery system that provides an improved delivery that Increases the speed, magnitude, and efficiency of delivery of the therapeutic, while avoiding problems with pill fatigue or pill burden.

### SUMMARY

A flexible oral therapy delivery system with enhanced permeability, and methods of manufacture, is disclosed. The flexible therapy delivery system is a ternary, oil-in-water emulsion containing three phases.

The three separate phases of this emulsion are generally comprised of:
a first aqueous phase that is hydrophilic
a second oil phase that is lipophilic
and a third activation or permeability enhancement phase.
   (Hydrophilic substances tend to be dissolvable in water; lipophilic substances tend to be dissolvable in lipids, such as oils and fats.)

This three-phase construction allows delivery of both water-soluble and oil-soluble compounds, with increased activation of either or both.

In some embodiments, the oil phase is comprised of a solubilizer and one or more oil-based therapy to be delivered. The solubilizer helps to transport the oil-based therapy within the ternary emulsion and deliver it.

In some embodiments, an oil capable of crossing the blood brain barrier and delivering one or more therapies across it is used. In an embodiment, the oil capable of crossing the blood-brain barrier is C-8 MCT (i.e., Medium Chain Triglycerides) caprylic acid triglyceride oil.

In some embodiments, a natural gum that has been spray dried into the form of a powder is provided. The gum acts as an overall emulsifier that allows the water and oil phases to cohabitate.

The third activation phase, in some embodiments, is comprised of four cellularly vital compounds capable of triggering active transport of material adjacent to cellular surfaces through the cellular surfaces. These four compounds are vital for cellular functioning as incentives to trigger active transport of medicinal material.

These compounds are:
1 sodium citrate.
2 potassium chloride
3 ascorbic acid (i.e., Vitamin C), and
4 magnesium glycinate.

Other compounds that are either essential or deficient to cells can also be used in place of any or all of these.

The presence of the four compounds in the third phase helps trigger active cellular transport. To activate cells, chemical elements and compounds that these cells are naturally and normally seeking, or there is a deficiency of, are provided by the activation (i.e., third) phase. All four of these are defined as electrolytes, needed for electrochemical balance.

Also, there is a membrane mechanism in animal cells known as the sodium-potassium pump that acts to intake potassium or sodium as needed, to maintain a specific level of both within the cell. In some embodiments, sodium and potassium are provided in the third phase to activate and utilize this sodium-potassium (i.e., Na—K) pump system and engage cell membrane transport. Because the Na—K pump mechanism seeks sodium and potassium, it acts to draw in the entire ternary emulsion within the cell to obtain the sodium and potassium within the emulsion

Further, the cells also naturally seek other compounds. These include magnesium and Vitamin C, contained by the other two third-phase compounds. In other words, rather than simple passive absorption by the cell, the combination herein activates the cell (i.e., cell activation) to actively seek and draw in the emulsion.

Other features of the third phase also provide additional permeability enhancement by changing the water activity of the ternary emulsion. The change in water activity from the third phase also provides antimicrobial properties, emulsion strength, emulsion stability, antioxidant properties, and Increased bioavailability.

If water activity of a substance can be reduced to under about 0.75 of 1.00, the substance cannot interact with or support microbes or microorganisms. The combination of compounds in the third phase reduces water activity of the ternary emulsion down to about 0.70. This low water activity level helps with both cell activation and preservation.

The oil-and-water-based phases are normally under constant pressure to separate from each other. The third activation phase, though, reduces the pressure of the oil and water phases to separate from each other. The water behaves a lot less like typical water, especially physically, so the oil-based phase does not try so hard to separate from it.

This results in a therapy that is absorbed with superior results to a nanoparticle, without the surfactants and other treatments necessary for a nanoparticle. Accordingly, cell absorption results as good or better than those that can be achieved for a nanoparticle, with the positives of a drinkable non-nanoparticle therapy. This reduction of the hydrophobic tendency of the oil portion also results in better delivery of the oil portion after the ternary emulsion has been taken into the cell.

The combination of the emulsion component, the specific oil selected, and the components contained in the third phase activate an increased uptake by active transport across mucosal surfaces. Each of these current compounds has delivery to human cells that is superior to most other compounds.

This improvement in emulsion technology, specifically the activation phase, activates the available delivery sites (i.e., cells/mucosa/junctions) for increased absorption of the therapy in the oral cavity, buccal (i.e., near, or relating to the cheek area), sublingual (i.e., area under the tongue), and mucosal surface (i.e., relating to the mucosa, which is the thin mucus-covered skin covering the inside surface of parts of the body) areas. These locations can be activated (i.e., incentivized) to increase permeability, bioavailability, and the speed of onset and magnitude (i.e., potency) of the molecules of the therapy.

In some embodiments, a manufacture process 20 the same or similar to the following can be used to make the three-phase delivery system. Generally, the three phases are each made and mixed with each other, then the final product is further processed.

In the first steps of the manufacturing process, the first phase, the aqueous phase, is created.

In a first step, in some embodiments, a natural gum, and in an embodiment a natural gum that has been spray dried into the form of a powder, is provided.

In a second step, the gum and water are combined. In some embodiments, at least one speed mixer is used to combine them, and in at least one embodiment, the type of speed mixer is a Dual Asymmetric Centrifuge (DAC).

In a third step, incremental amounts of water are added until the gum is fully hydrated. This is a technique known as “thick phasing.” This “thick phasing” is done in gradual steps, which keeps the product physically broken down. The sheer of particle collisions with the speed mixer causes heat, which also helps with smoother mixing

In a fourth step, the aqueous phase is pasteurized. This phase is typically pasteurized at least twice for antimicrobial safety, and stability—here, and then later in the process.

In the next steps, the second phase, the oil-based phase, is created and combined with the first phase.

In a fifth step, oil soluble active compounds are combined with C-8 MCT caprylic acid triglyceride oil to create the second phase.

In a sixth step, the first and second phases are combined and mixed. In some embodiments, they are combined using the speed mixer, and typically mixed at least twice.

In the next steps, the third phase, the activation phase, is created and combined with the first and second phases.

In a seventh step, sodium citrate is added to the first two phases as an emulsion strength enhancer. This begins the addition of the third phase to the first two phases.

In an eighth step, the third phase is completed by adding the remaining components: potassium chloride, ascorbic acid (Vitamin C), and magnesium glycerinate. These are also typically mixed in with the speed mixer.

In a ninth step, the final mixed product is pasteurized, as in a previous step.

In a tenth step, the cooled product is stored in refrigeration until needed. The final product, the oral therapy, exists in a neutrality between oil and water portions. It can be used successfully for delivering oil-soluble or water-soluble therapies.

This invention provides an improved oral delivery system that delivers a therapy past cell walls and where needed with increased efficiency, and an innovative method of manufacturing it.

### BRIEF DESCRIPTION OF THE DRAWINGS

FIG. 1 is a schematic diagram of a typical therapy delivery system in use.

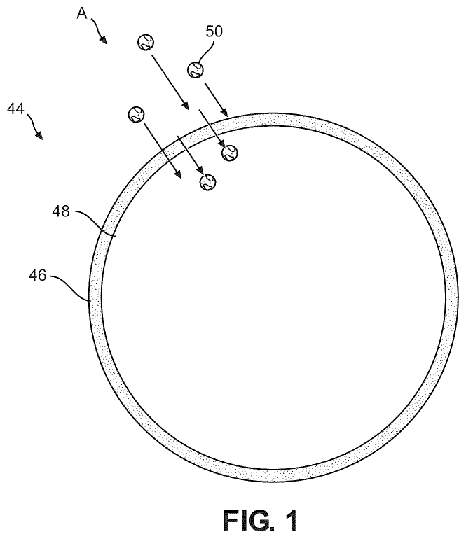

FIG. 2 is a schematic diagram showing an embodiment of the three-phase emulsion of the invention.

FIG. 3 is a schematic diagram showing an embodiment of the invention in progress.

FIG. 3A is a schematic diagram showing an embodiment of the invention showing delivery completed.

FIG. 4 is a schematic flow diagram of an embodiment showing a method of manufacture.

### DETAILED DESCRIPTION OF PREFERRED EMBODIMENTS

A number of objects, features, and advantages of the invention will become apparent from a consideration of the following detailed description and the accompanying drawings. The following descriptions are made referring to the figures, wherein like reference number refers to like features throughout this description. It is to be understood some portions or components may not be visible in some figures.

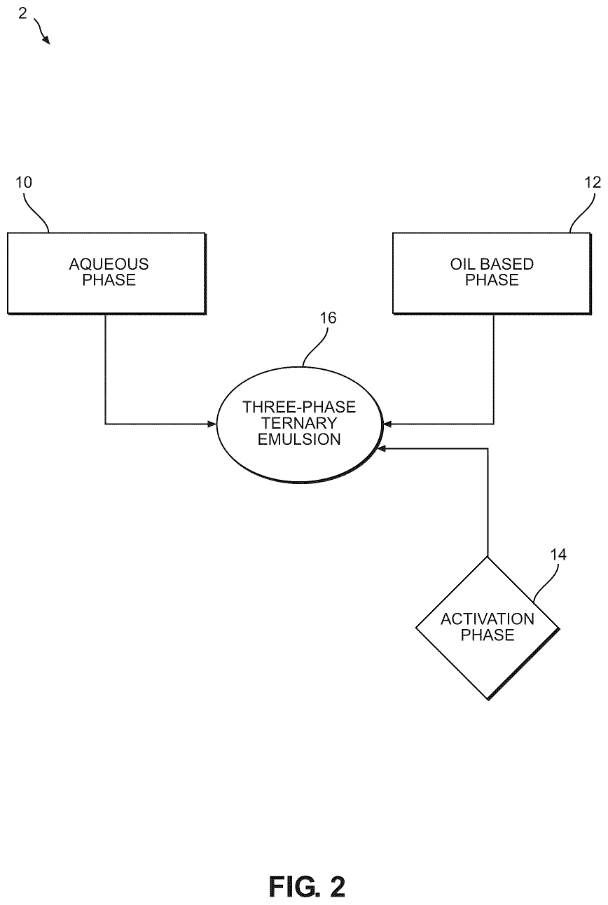

Turning to FIG. 2, an embodiment of an improved emulsion-based therapy delivery system 2 is shown.

The flexible therapy delivery system 2 herein, in pharmaceutical terminology, would be described as a ternary, oil-in-water emulsion 16, containing three phases.

The three separate phases of this emulsion are generally comprised of:
a first aqueous phase 10 that is hydrophilic
a second oil phase 12 that is lipophilic
and a third activation or permeability enhancement phase 14.
   (Hydrophilic substances tend to be dissolvable in water; lipophilic substances tend to be dissolvable in lipids, such as oils and fats.)

This three-phase construction allows delivery of both water-soluble and oil-soluble compounds, with increased activation of either or both.

In some embodiments, the oil phase is comprised of a solubilizer and one or more oil-based therapy to be delivered. The solubilizer acts as a kind of solvent to the oil-based therapy and helps to transport the oil-based therapy within the ternary emulsion and deliver it. The solubilizer can be a number of oils or combination of oils, such as, e.g., olive oil or canola oil.

In some embodiments, an oil capable of crossing the blood-brain barrier and delivering one or more therapies across it are used.

In an embodiment, the oil capable of crossing the blood-brain barrier is C-8 MCT (i.e., Medium Chain Triglycerides) caprylic acid triglyceride oil. MCTs are additionally known as Caprylic/Capric Triglycerides. C-8 MCT. This type of oil is typically derived from the distilled fatty acid fractions of palm kernel oil. This oil is particularly effective at crossing the blood-brain barrier, which this is one of the purposes for which it is included.

Among the oil-based therapies that can be solubilized by the solubilizer, and in an embodiment the C-8 MCT is a cannabinoid.

In some embodiments, a natural gum, in some embodiments a gum that has been spray dried into the form of a powder is provided. The gum acts as an overall emulsifier that allows the water and oil phases to cohabitate.

The third activation phase 14, in some embodiments, is comprised of four cellularly vital compounds (i.e., incentives) capable of triggering active transport of material adjacent to cellular surfaces through the cellular surfaces. These four compounds are vital for cellular functioning as incentives to trigger active transport of medicinal material.

These compounds are:
1 sodium citrate
2 potassium chloride
3 ascorbic acid (Vitamin C), and
4 magnesium glycinate.

Regarding these four compounds;

Sodium citrate is the tri-sodium salt of citric acid. Sodium citrate can act as a secondary emulsifier that increases (i.e., tightens) the emulsification and stability, and can increase overall oil loading. This stabilizes (i.e., tightens) the emulsion, allowing the emulsion to carry more oil. Sodium citrate helps lower water activity, and is a source of sodium. Sodium helps activate the cells 44 via the sodium potassium pump 48.

Potassium chloride is a naturally occurring salt that is sometimes used as a mineral supplement to treat low potassium levels. It provides a source of potassium for the sodium potassium pump 48 and acts to lower water activity, also serving as an antimicrobial.

Ascorbic acid (Vitamin C) is a water-soluble vitamin found naturally in citrus fruits and a number of vegetables. Ascorbic acid binds water and lowers water activity, plus is antimicrobial. It is also an antioxidant, preventing oxidation reactions.

Magnesium glycinate is an orally available magnesium salt of glycine. Magnesium is highly sought by cells, so it helps with cell activation. It also helps lower water activity and is antimicrobial.

Other chemical elements or compounds that are either essential to, or deficient in, cells can also be used in place of any or all of these. In other embodiments, other water-soluble therapeutics that are essential for cell functioning can be substituted. These are typically other compounds or elements that the cells would naturally seek. As will be discussed herein, the more valuable a component is to the cell, the more naturally a cell will seek it, and, accordingly, allow an emulsion of which the component is a part, into the cell.

In other embodiments, the water-soluble therapeutic or therapeutics can be tailored to a specific group of consumers with a specific need for, or known deficiency of, an element or compound. If a specific type of consumer, or a group of consumers with a specific condition, are known to have a shortage of one or more particular elements or compounds, this one or more elements or compounds can be added into the activation phase tailored to that cellular shortage or need. This can make this activation phase even more sought and absorbed by cells. This can also provide opportunities to add nuance to formulations.

In a specific example, it is known that consumers in the United States, who tend to consume more processed food than much of the world, are more likely to have a higher need for magnesium, with cells seeking magnesium more than usual. Accordingly, when magnesium is part of the activation phase, there is a good chance this will have even higher cellular absorption among therapeutic consumers in the US.

The purposeful ratio of the four compounds relative to each other for maximum effectiveness can vary, based on factors such as the type of consumer, condition to be treated, and the nature of the therapy.

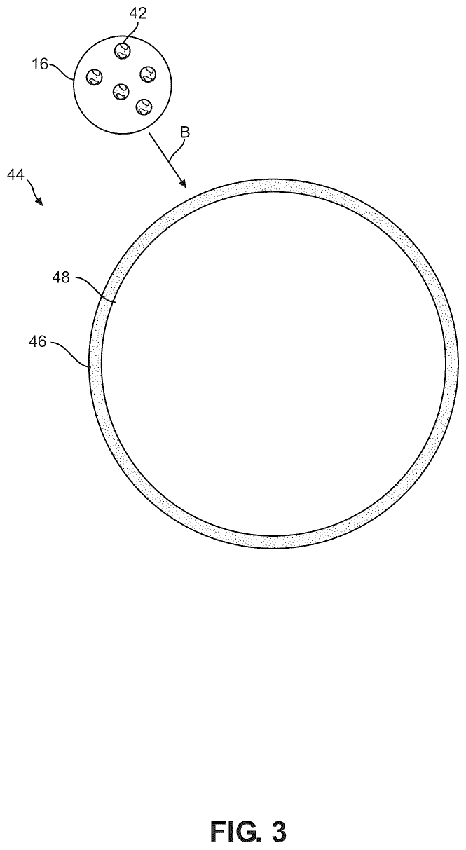

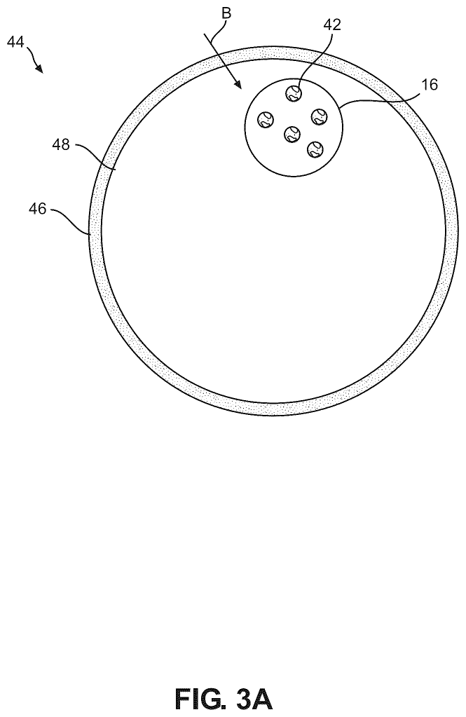

Turning to FIG. 3-3A, the active transport mechanism of the ternary emulsion 16 into cells 44 is shown.

Each of these compounds has delivery to human cells that is superior to most other compounds. The presence of the four compounds in the third phase helps trigger active cellular transport. To activate cells 44, elements and compounds the cells 44 are naturally and normally looking for, or there's a deficiency of, are provided by the activation (i.e., third) phase. For example, cells seek sodium, potassium, magnesium, and Vitamin C. These are necessary for life and sought after by cells at all times. Further, all four of these are defined as electrolytes, needed for electrochemical balance.

Particularly, sodium and potassium are provided to the cells through the sodium-potassium (i.e., Na—K) pump 48 that is present in animal cells. Animal cells contain a membrane mechanism known as the sodium-potassium pump 48 that acts to intake potassium or sodium, as needed, to maintain a specific level of both within the cell 44. Here, the Na—K pump is represented as 48. In the third phase, sodium and potassium are provided to activate and utilize this pump system and engage cell membrane transport. Because the Na—K pump mechanism seeks sodium and potassium, particularly potassium, it acts to draw in the entire ternary emulsion 16 within the cell to obtain the sodium and potassium within, as indicated by arrow set B, and as shown specifically in FIG. 3. Turning to FIG. 3A, the successful transport of the ternary emulsion 16 to within the cell 44 is shown.

Further, the cells 44 also naturally seek other compounds. These include magnesium and Vitamin C, contained by the other two third-phase compounds. Arrow set B also shows these being drawn into the cell 44. In other words, rather than simple passive absorption by the cell 44, the combination herein activates the cell 44 (i.e., cell activation) to actively seek and draw in the emulsion 16.

Other features of the third phase also provide additional permeability enhancement by changing the water activity of the ternary emulsion 16. The change in water activity from the third phase also provides antimicrobial properties, emulsion strength, emulsion stability, and antioxidant properties, and increased bioavailability.

As previously mentioned, if water activity of a substance can be reduced to under about 0.75 of 1.00, the substance cannot interact with or support microbes or microorganisms. The combination of compounds in the third phase reduces water activity of the ternary emulsion 16 down to about 0.70. This low water activity level helps with both cell activation and preservation.

The oil- and water-based phases 10, 12 are normally under constant pressure to separate from each other. The third activation phase 14, though, reduces the pressure of the oil and water phases to separate from each other. The water behaves a lot less like typical water, especially physically, so the oil-based phase 12 does not try so hard to separate from it. This places the emulsion in a neutrophilic, or close to it, state, in which the emulsion 16 does not favor water or oil (neither hydro or lipophilic). It is to be understood that the term for this state can be either neutrophilic or neutral philic. The invention herein results from a form of reactionless chemistry—chemistry constructed to reduce or limit a reaction, such as the separation of water and oil. This neutral philic state, along with the cell 44 actively seeking the compounds, results in cell activation and absorption of the ternary emulsion 16. This results in a therapy that is absorbed with superior results to a nanoparticle, without the surfactants and other treatments necessary for a nanoparticle. Accordingly, cell absorption results can be reached that are as good or better than those that can be achieved for a nanoparticle, with the positives of a drinkable non-nanoparticle therapy.

This reduction of the hydrophobic tendency of the oil portion also results in better delivery of the oil portion after the ternary emulsion 16 has been taken into the cell 44.

It is important to note that the four compound components of the third phase herein are designed such that each has multiple uses, generally, most or all of the compounds assist with. For example, instead of preservatives, these chemicals help lower the water activity to a point where preservatives are no longer needed and provide a source of something the cell 44 seeks naturally, helping with cell activation.

The combination of the emulsion component, the specific oil selected, and the components contained in the third phase activate an increased uptake by active transport across mucosal surfaces. Each of these current compounds has delivery to human cells that is superior to most other compounds.

This improvement in emulsion technology, specifically the activation phase, activates the available delivery sites (i.e., cells/mucosa/junctions) for increased absorption of the therapy in the oral cavity, buccal (i.e., near, or relating to the cheek area), sublingual (i.e., area under the tongue), and mucosal surface (relating to the mucosa, which is the thin mucus-covered skin covering the inside surface of parts of the body) areas. These locations can be activated (i.e., incentivized) to increase permeability, bioavailability, and the speed of onset and magnitude (i.e., potency) of the molecules of the therapy.

The process and components herein have been selected and developed for functionality and efficacy. Users of this invention will receive faster onset, increased bioavailability, and greater magnitude of therapy, particularly over lower efficiency solid form therapies. Additionally, nanoparticles have an early onset, but it leaves earlier, making it less efficient. The therapy delivery system 2 of the invention herein tends to begin interaction early, with greater efficiency, with the advantage of staying in the body longer.

This delivery system addresses other issues as well. Current emulsion systems, particularly cannabinoid-based emulsion components, tend to be somewhat sensitive to work with or handle. They tend to have weaknesses of instability, tendency to separate, inability to be pasteurized, inability to endure freeze-thaw cycles, and a need for significant reformulating for different active ingredients. These are issues whether they are water or oil soluble.

The formulation herein addresses these issues, offering, among other advantages, added stability of the compound. It also has less tendency to separate, can be pasteurized, can endure freeze-thaw cycles, and doesn't need significant reformulating for different active ingredients, whether they are water or oil soluble.

The oral delivery systems, in some embodiments, can be used for pharmaceutical, nutraceutical, botanical, nootropic, and consumer supplements. The oral therapy herein can be useful to a number of parties, including patients, doctors, hospitals, and other medical facilities, pharmacies, and manufacturers of various therapies.

The physical form of the final product is typically a thick, viscous but pourable and drinkable liquid. In beverage terminology, this invention, in some embodiments, is a Concentrate or Beverage Syrup. The Invention can be further customized to include any beverage flavor. The invention is compatible with the beverage flavors, bitter blockers, flavor masking agents, weighting agents, natural colorings, and sweeteners utilized in the beverage industry. It can be adapted to taste, for instance, like a cola, milkshake, or lemonade. Many beverage flavors can be adapted using standard beverage components and processes.

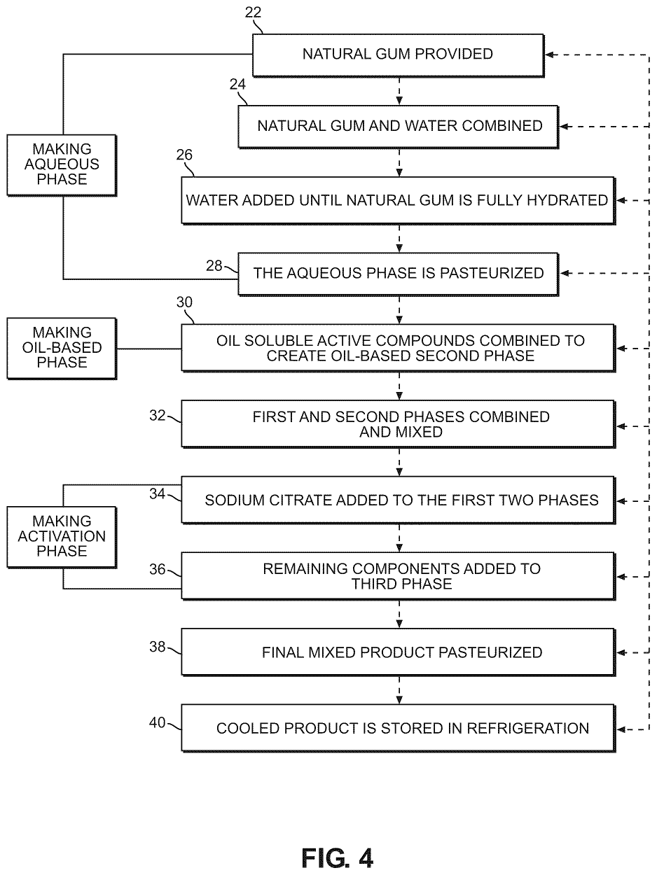

Turning to FIG. 4, in some embodiments, a manufacture process 20 the same or similar to the following can be used to make the three-phase delivery system. Generally, the three phases are each made and mixed with each other, then the final product is further processed. As indicated by the arrows, the steps can be performed in a different order, simultaneously, or individual steps may be skipped or moved.

In the first steps of the manufacture process 20, the first phase, the aqueous phase, is created.

In a first step, in some embodiments, the natural gum, and in this embodiment, a natural gum that has been spray dried into the form of a powder, is provided 22.

There are a variety of natural biopolymers and gums that can be used to help create the emulsion herein. In some embodiments, these can be a high oil load carrying biopolymer or gum. These can include, for a few examples, a Xanthan gum, Guar gum, or a Carrageenan gum.

In this embodiment, the natural gum is a gum Arabic in powdered form (derived from the hardened sap of an acacia tree). Natural gum Arabic is used in this embodiment because it is the highest possible oil-loading gum for the formulations herein. In other words, this gum can carry the highest known amount of oil for the ternary emulsion herein. Though the natural gum is typically in the form of a powder, there is still technically a miniscule amount of water in it. However, there is so little water, the water activity is only around 0.30 or less with effectively no water available for microbial activity such as spoilage. The little water present is bound to the powder and not available for interaction. This, though, can change as more water is added.

In a second step, the gum and water are combined 24. A machine is typically used, and in some embodiments, at least one speed mixer is used to combine them, and in at least one embodiment, the type of speed mixer is a Dual Asymmetric Centrifuge (i.e., DAC). The gum and water are mixed using the speed mixer for at least one cycle. In a further embodiment, the speed mixer is used for four cycles.

Other high-speed mixing machines known in the art can be used, such as, e.g., a high-sheer homogenizer, colloid mill, or micro-fluidizer. As a specific example, this type of mixing is often performed in the beverage industry with a high-sheer homogenizer and can be performed with one in some embodiments herein. However, a speed mixer does offer a number of advantages over other mixing machines. The other types of machines capable of mixing this formulation listed herein tend to be high cost, requiring a higher capital investment than a speed mixer. These other machines tend to be very large and prohibitively expensive pieces of equipment used for mixing very large batches of beverages. The speed mixer makes making smaller, more individualized batches easier. This provides more flexibility, more ability to provide customized or individualized batches, and more modularity with batches.

Further, these larger, commercial-grade machines can take a long time to repair when needed. In addition, speed mixers allow for more and smaller batches. Accordingly, if a batch is defective in some way, replacing the batch is less costly in work and expense.

In addition, because a speed mixer typically relies on oppositional spinning of portions of the batch, with natural collision and sheer forces, rather than using blades, cleaning and sterilization of the speed mixer tends to be much simpler, easier, and less time intensive.

It has been found that the speed mixer can save a lot of processing time, and save time gradually heating the mixture, because the sheer speed of the speed mixer generates the right amount of heat, if used properly, to heat the product as it mixes. Accordingly, no separate heating of the product is needed. The use of a speed mixer, and method of use, in the manufacture of this substance represents a unique and significant technological step forward.

A DAC, or speed mixer, is typically used in distant high-tech industries like ceramics and aerospace. It is difficult to repair, expensive, and appears to have no presence in the beverage industry.

In operation, the speed mixer, or DAC, can run in a pattern suitable for breaking up a solid substance and mixing it with liquid. In this embodiment, the speed mixer typically has two centrifuges that together run in a pattern shapes somewhat like an infinity loop ∞. In this embodiment, the two centrifuges of the speed mixer run in opposite planes, creating a pair of loops running in opposite directions. For visualization, millions of particles are running into the infinity double loop pattern where the millions of particles are colliding, creating the heat to solubilize the gum, plus smashing into each other to physically break them down.

In a third step, incremental amounts of water are added until the gum is fully hydrated 26. This is a technique known as “thick phasing.” Hygroscopic (i.e., high affinity for absorbing water) gums, such as the ones involved here, have a bloom factor. When water is added, they absorb water and have a swelling reaction. This swelling needs to be controlled. If the mix is too fast, the gum and water will clump, resulting in an uneven, unusable mix of the two-basically, a clumpy mess. To avoid this, water is gradually added to the spray-dried powder while they are gradually mixed using the speed mixer. This “thick phasing” is done in gradual steps, which keeps the product physically broken down. The sheer number of particle collisions with the speed mixer causes heat, which also helps with smoother mixing and managing bloom.

Water is added at least three times. The first cycle of added water captures most gum particles. Then more water is added to complete swelling, to a semi-liquid. Then more water is added to the mix to complete conversion to a liquid. Eventually this creates a full aqueous phase.

In a fourth step, the aqueous phase is pasteurized 28. Powders tend to have high rates of bacteria, yeast, mold, fungus, and microbes. It is pasteurized for about two hours, cooled at room temperature, then typically stored in refrigeration at around 4 degrees Celsius. This phase is typically pasteurized at least twice for antimicrobial safety, and stability—here, and then later in the process. This mixture serves a dual function, as both the aqueous phase and a primary emulsifier, to assist with mixing this first phase with the second phase. Most emulsion products will come apart under pasteurization, making pasteurization difficult or impossible. The therapy delivery system 2 herein does not tend to come apart because the lack of water activity holds it together by negating the hydrophobic forces that would normally pull emulsion delivery systems apart. Further, the delivery system herein 2 can be frozen and thawed, unlike other therapies, for similar reasons.

In the next steps, the second phase, the oil-based phase, is created and combined with the first phase.

In a fifth step, oil-soluble active compounds are combined with C-8 MCT caprylic acid triglyceride oil to create the second phase 30. A number of oil-soluble compounds can be used, including Vitamin D, Omega 3, oil-soluble vitamins. In an embodiment, the oil-soluble active compounds can be one or more cannabinoids.

In a sixth step, the first and second phases are combined and mixed 32. In some embodiments, they are combined using the speed mixer and typically mixed at least twice.

In the next steps, the third phase, the activation phase, is created and combined with the first and second phases.

In a seventh step, an activation compound or element, here sodium citrate, is added to the first two phases as an emulsion strength enhancer 34. They are typically then mixed with the speed mixer. This begins the addition of the third phase 14 to the first two phases 10,12.

In an eighth step, the third phase is completed 36 by adding the remaining activation components: potassium chloride, ascorbic acid (Vitamin C) and magnesium glycerinate. These are also typically mixed in with the speed mixer.

In a ninth step, the final mixed product is pasteurized 38, as in a previous step. In this pasteurization step, it is typically pasteurized for about one hour in a shorter flash pasteurization, and then cooled. This second later pasteurization is performed because lots of things have been combined since the first pasteurization that are probably not sterile.

In a tenth step, the cooled product is stored in refrigeration until needed 40. Typically, the product is stored in refrigeration at about 4 degrees Celsius. The substance can be frozen and thawed, as necessary, and remain stable.

The final product, the oral therapy, is basically amphiphilic, or amphipathic, having a balance between hydrophobic and hydrophilic. The therapy delivery system 2 exists in a neutrality between oil and water portions. It can be used successfully for delivering oil-soluble or water-soluble therapies.

As examples, Vitamin D, Omega 3, or other oil-based therapy can be placed in the oil-based phase 10 within the ternary emulsion 16 and delivered. Oran aqueous-based therapy such as, e.g., any of the water-soluble B-vitamins, Vitamin-C, or other water-soluble therapy, can be placed in the aqueous phase 12 and delivered. This can be used, respectively, for water-soluble pharmaceuticals, oil-soluble pharmaceuticals, nutraceuticals, nootropics, or any of the active compounds in the health supplement field.

In another example, cannabinoids tend to be lipophilic as they are generally comprised of oils. When mixed with water or another aqueous portion, the oils tend to be hydrophobic, separate, and accumulate against the side of the container. This means that cannabinoids are difficult to mix effectively within an emulsion, which makes them difficult and inefficient to deliver effectively as a therapy. However, within the ternary emulsion 16 herein, cannabinoids are not nearly as hydrophobic, as the aqueous portion has a low water activity, behaving less like water. Accordingly, they can be part of the oil-based portion and are much more effectively delivered with the therapy delivery system 2 herein.

This invention provides an improved oral delivery system that delivers a therapy past cell walls where needed with increased efficiency, and an innovative method of manufacturing it. The manufacturing process also results in improved stability.

It is to be understood that while certain forms of the present invention have been Illustrated and described herein, the expression of these individual embodiments is for illustrative purposes and should not be seen as a limitation upon the scope of the invention. It is to be further understood that the invention is not to be limited to the specific forms or arrangements of parts described and shown.

1. An oral therapy delivery system, comprised of a three-phase ternary emulsion,
wherein the three-phase ternary emulsion is comprised of:
a first hydrophilic aqueous phase,
a second lipophilic oil-based phase,
and a third activation phase,
and wherein the third activation phase is comprised of at least one element or compound capable of triggering active transport of a therapy through a cellular surface and into a cell,
and wherein the oral therapy delivery system is configured to successfully deliver at least one oil soluble therapy, water soluble therapy, or both.

2. An oral therapy delivery system according to claim 1,
wherein the second oil-based phase is further comprised of a C-8 MCT caprylic acid triglyceride oil.

3. An oral therapy delivery system according to claim 1,
wherein the second oil phase is comprised of a solubilizer and at least one oil-based therapy.

4. An oral therapy delivery system according to claim 3,
wherein the at least one oil-based therapy is a cannabinoid.

5. An oral therapy delivery system according to claim 1,
wherein the first aqueous phase is further comprised of a natural gum.

6. An oral therapy delivery system according to claim 1,
wherein the third activation phase is further comprised of:
sodium citrate,
potassium chloride,
ascorbic acid, and
magnesium glycinate.

7. An oral therapy delivery system according to claim 1,
wherein the therapy delivery system is generally amphiphilic, existing in a neutrality between oil-based and aqueous phases.

8. An oral therapy delivery system according to claim 1,
wherein the ternary emulsion is configured to deliver a water-soluble pharmaceutical, an oil-soluble pharmaceutical, nutraceutical, botanical, nootropic, consumer supplement, cannabinoid, or a combination of these.

9. An oral therapy delivery system according to claim 1,
wherein the therapy delivery system is in the form of a concentrate or beverage syrup,
and wherein the therapy delivery system can be configured to include a beverage flavor, bitter blocker, flavor masking agent, weighting agents, natural coloring, sweeteners, or any combination of these.

10. A method of providing a three-phase ternary emulsion comprising the steps of:
providing a first hydrophilic aqueous phase,
providing a second lipophilic oil-based phase,
and a third activation phase,
wherein the first aqueous phase is further comprised of at least one natural gum,
wherein the second oil-based phase is further comprised of a C-8 MCT caprylic acid triglyceride oil,
and wherein the third activation phase is further comprised of:
sodium citrate,
potassium chloride,
ascorbic acid, and
magnesium glycinate.

11. A method of manufacturing an oral therapy delivery product comprising the steps of:
providing a first aqueous phase by:
providing at least one biopolymer or natural gum,
combining the gum and water using a machine, and
providing a second oil-based phase by:
providing at least one oil or oil-soluble compound configured to be combined with at least one oil-soluble active compound, and
combining the first and second phases,
providing a third activation phase by:
providing a first activation element or compound and combining it with the first two phases,
and providing any remaining activation elements or compounds,
and combining them with the first two phases.

12. A method of manufacturing an oral therapy delivery system according to claim 11, wherein the biopolymer or natural gum is a gum Arabic, Xanthan gum, Guar gum, a Carrageenan gum, or a combination of these.

13. A method of manufacturing an oral therapy delivery system according to claim 11, wherein the machine is at least one speed mixer, high-sheer homogenizer, colloid mill, micro-fluidizer, or a combination of these.

14. A method of manufacturing an oral therapy delivery system according to claim 13, wherein the machine is at least one speed mixer, and the speed mixer is a Dual Asymmetric Centrifuge,
and wherein the gum and water are combined using the speed mixer for at least one cycle.

15. A method of manufacturing an oral therapy delivery system according to claim 11, providing the further step of:
after the gum and water are combined using a machine, gradually adding further incremental amounts of water until the biopolymer or natural gum is fully hydrated.

16. A method of manufacturing an oral therapy delivery system according to claim 11, providing the further step of pasteurizing the first aqueous phase after the biopolymer or gum is combined with water.

17. A method of manufacturing an oral therapy delivery system according to claim 16, providing the further step of pasteurizing the product a second time after the three phases have been combined.

18. A method of manufacturing an oral therapy delivery system according to claim 11,
wherein the at least one oil or oil-soluble compound is a C-8 MCT caprylic acid triglyceride oil,
and providing the further step of combining the C-8 MCT caprylic acid triglyceride oil with at least one oil-soluble active compound.

19. A method of manufacturing an oral therapy delivery system according to claim 18, wherein the at least one oil-soluble active compound is Vitamin D, Omega 3, an oil-soluble vitamin, one or more cannabinoids, or a combination of these.

20. A method of manufacturing an oral therapy delivery system according to claim 11,
wherein the first activation compound is sodium citrate,
and wherein the any remaining activation elements or compounds are potassium chloride, ascorbic acid, and magnesium glycerinate.
